(19) United States
(12) Patent Application Publication
(10) Pub. No.: US 2001/0039012 A1
(43) Pub. Date: Nov. 8, 2001

(54) Methods for diagnostic screening

(76) Inventor: Stanley N. Lapidus, Bedford, NH (US)

Correspondence Address: TESTA, HURWITZ & THIBEAULT, LLP, HIGH STREET TOWER, 125 HIGH STREET, BOSTON, MA, 02110, US

(21) Appl. No.: 09/748,428

(22) Filed: Dec. 26, 2000

Related U.S. Application Data
(63) Continuation of application No. 09/332,331, filed on Jun. 14, 1999, now abandoned.

(51) Int. Cl. C12Q 1/68

(52) U.S. Cl. 435/6

(57) ABSTRACT

Methods are presented for mass screening of patient populations for indicia of disease, infection, or predisposition to disease.

### BACKGROUND OF THE INVENTION

Molecular disease diagnostic methods have become of interest with the advent of techniques such as polymerase chain reaction and restriction fragment length polymorphism analysis. The ability to detect nucleic acid alterations that are indicative of disease provides a powerful tool in diagnosis and treatment. Typical assays identify a gene, or a mutation in a gene, that is thought to be associated with a disease. A popular method for identifying disease-associated mutations involves the detection of restriction fragment length polymorphisms in order to identify those of medical significance. Other methods have focused on multiple mutation detection using multiple sequence-specific probes and detecting those that hybridize to DNA in patient samples in order to correlate DNA sequences with disease status.

Molecular diagnostic techniques typically are expensive, and are not cost-effective for routine diagnosis, especially of conditions that have a low incidence in the population. However, such techniques may provide the best opportunity for early disease diagnosis. In many cases, early disease diagnosis makes a significant difference in a patient's prognosis, and the course of treatment prescribed. Cancer is an example of a disease that, in many cases, is treatable if diagnosed early. Since many cancers are associated with genomic mutations (e.g., ranging from loss of heterozygosity to point mutations) that are not easily and inexpensively detected, many patients remain undiagnosed until supramolecular indicia of the disease are evident.

There is a need for efficient, relatively inexpensive diagnostic procedures that enable disease screening. Such methods are provided by the present invention.

### SUMMARY OF THE INVENTION

The present invention provides methods for screening populations of patients for indicia of disease. According to methods of the invention, combined tissue or body fluid samples from a plurality of patients are analyzed for the presence of one or more disease markers. If no targeted disease marker is found in the combined sample, all patients making up the combined sample are diagnosed as negative for the disease or diseases targeted in the combined sample analysis.

If one or more disease marker is (are) detected in the combined sample, one or more subsamples containing tissue or body fluid from a subpopulation of patients comprising the combined sample are analyzed for the presence of the marker(s). The process of constructing subsamples continues serially until the presence of a disease marker is unambiguously associated with a patient sample from which it is derived. Thus, if a disease marker is detected in a combined sample, two or more new subsamples are prepared, and analysis is conducted on each of the two new samples. If a disease marker is found in only one of the two new subsamples, patients making up the subsample in which a marker was not found are determined to be negative for the disease(s) being diagnosed. Additional subsamples from patients making up the subsample in which a marker is detected are then made and tested. This process continues until a patient sample or samples is (are) identified as possessing the marker or markers to be detected.

Methods of the invention provide rapid and efficient means for diagnosing disease in a plurality of patient samples without requiring analysis of each patient sample. Such methods reduce costs associated with serial analysis of individual samples, and allow essentially simultaneous diagnosis in a plurality of patient samples. Methods of the invention are applicable to any screening assay. Methods of the invention are especially useful for diagnostic screening assays, including molecular assays, cytologic assays, immunoassays, and any other assay in which there exists a marker associated with a disorder the detection of which is desired.

As used herein, a disease marker is a chemical entity that can be associated with disease. The marker may be indicative of the actual presence of disease, may indicate the propensity for disease, or may be indicative of the stage of a disease. In addition, the marker may be associated with a syndrome (e.g., AIDS) or a condition that is predisposing to a disease or syndrome (e.g., HIV). Markers detected in methods of the invention may identify an infection, whether it has manifested itself in disease or not, a parasite, or a genetic alteration that is associated with, or predisposing to, a disease. Accordingly, a disease marker detected in methods of the invention may be a nucleic acid (mutant or wild-type); a protein or peptide, including an antibody; a hormone; a sugar; a carbohydrate; a polymer; or a synthetic or composite compound produced by association or reaction with a marker (e.g., a conjugate or a detection moiety associated with a marker of an enzymatic product). Alternatively, a disease marker may be a visual marker of disease (e.g., dense cellular nuclei).

In a preferred embodiment, methods of the invention comprise combining tissue or body fluid samples from a plurality of patients and analyzing the samples for markers indicative of cancer. In a highly-preferred embodiment, such markers are genetic markers, such as mutations associated with cancer or with the propensity for cancer. For example combined patient samples are analyzed for mutations in a tumor suppresser gene, such as p53, using methods capable of detecting an alteration in the gene that impairs its ability to regulate the cell cycle. Methods such as sequence-specific capture are used for such analysis. More preferably, enumerative methods for the detection of early stages of cancer, such as those described in U.S. Pat. No. 5,670,325, incorporated herein by reference, are used. If no markers indicative of cancer are found in this first analysis, patients whose samples comprise the combined sample are determined to be negative. If a marker is found in the combined sample, the combined sample is serially subdivided until the patient or patients having the marker can be identified.

Methods of the invention also are useful for screening donated tissue or body fluid samples. For example in cases of blood donation, methods of the invention provide an economical and rapid means for screening samples for hepatitis, HIV, and other infectious agents.

Methods of the invention are useful for simultaneously screening a sample for multiple disease markers. That embodiment is especially useful when all the markers to be screened are expected to occur only rarely in the population being analyzed (rare event markers). Methods of the invention are useful to perform assays to simultaneously or sequentially detect multiple rare event markers. If any of the rare event markers are found in the initial screen, samples are subdivided in the manner described above, and subsamples are analyzed until each detected marker can be associated with a particular patient. Alternatively, in, for example, blood screening, combined samples having a disease marker can be immediately discarded if an additional measure of security against contaminating the sample bank is desired.

Methods of the invention are useful for screening samples typically used for cytological analysis. For example, methods of the invention are used to screen pap smear samples combined from multiple patients. Initial screening may be performed by visual analysis of the combined sample, or by chemical assay. In either case, if the indicia of cancer are found, the combined sample is divided into two or more samples for analysis. Any of those subdivided samples in which indicia of disease are found, are further subdivided for analysis. This process continues seriatim until the disease marker(s) is(are) identified with one or more patients.

The particular assay used in methods of the invention depends upon the marker to be detected. In general, an assay must be sensitive enough to detect the appropriate marker in a combined sample. Accordingly, assays that use detectable labels, such as radio-isotopes, fluorescent markers, or colorimetric markers are especially useful. However, to be effective, an assay must allow detection of the event or events in the sample that are indicative of the disease for which screening is desired. In a particularly-preferred embodiment, assays used in methods of the invention are suitable to detect the presence of a marker in a combined sample, wherein the marker is indicative of a disease or condition that is infrequent in the population being screened.

In a preferred embodiment, a combined sample comprises samples obtained from between 2 and about 1000 patients. In another preferred embodiment, a combined sample comprises samples obtained from between 2 and about 500 patients. In a highly-preferred embodiment, a combined sample comprises samples obtained from between two and about 100 patients. The number of subsamples constructed once a marker is detected in a combined sample is limited by the practitioner's choice, financial considerations, and the ability of the selected assay to detect a target marker.

By their nature, methods of the present invention are practiced on samples from any tissue or body fluid source. Particularly-preferred samples include sputum, blood, stool, biopsy tissue, urine, cerebrospinal fluid, saliva, hair, and skin. The particular sample used in practice of the invention depends on the disease for which detection is desired, and the marker to be detected.

Detection of disease markers is carried out by any applicable method. For example, antibodies that bind to markers of interest are useful. On a molecular level, a disease marker may be a single nucleotide polymorphic variant that is associated with a disease or with a propensity for disease. Such variant nucleic acids are detected by molecular assays. A preferred molecular assay comprises counting numbers of a nucleic acid expected to be present in a sample and comparing that number to the number actually determined to be in the sample. Such methods are useful for detecting a disruption in genomic stability, such as loss of heterozygosity or another mutation, that is a marker for disease.

In a preferred embodiment, methods of the invention comprise screening to detect a single-nucleotide polymorphic variant that is indicative of disease. Accordingly, a combined sample comprising like tissue or body fluid from a plurality of patients to be tested is obtained. A preferred method of testing for the presence of a single-nucleotide variant that is indicative of disease is to conduct a single base extension assay. Such an assay is performed by annealing an oligonucleotide primer to a complementary nucleic acid, and extending the 3′ end of the annealed primer with a chain terminating nucleotide that is added in a template directed reaction catalyzed by, for example, a DNA polymerase. The chain-terminating nucleotide will identify the single base by complementarity. Alternatively, the chain-terminating nucleotide is added downstream of the 3′ end of the primer, and the single nucleotide is identified as a unique intervening base between the 3′ end of the primer and the chain-terminating nucleotide.

The selectivity and sensitivity of a single base primer extension reaction are affected by the length of the oligonucleotide primer and the reaction conditions (e.g., annealing temperature, salt concentration). The selectivity of a primer extension reaction reflects the amount of exact complementary hybridization between an oligonucleotide primer and a nucleic acid in a sample. A highly-selective reaction promotes primer hybridization only to nucleic acids with an exact complementary sequence (i.e. there are no base mismatches between the hybridized primer and nucleic acid). In contrast, in a non-selective reaction, the primer also hybridizes to nucleic acids with a partial complementary sequence (i.e. there are base mismatches between the hybridized primer and nucleic acid). In general, parameters which favor selective primer hybridization (for example shorter primers and higher annealing temperatures) result in a lower level of hybridized primer. Therefore, parameters which favor a selective single-base primer extension assay result in decreased sensitivity of the assay.

In a preferred method for detection, at least two cycles of a single-base extension reaction are conducted. By repeating the single-base extension reaction, the signal of a single-base primer extension assay is increased without reducing the selectivity of the assay. Cycling increases the signal, and the extension reaction can therefore be performed under highly selective conditions (for example, the primer is annealed at about or above its Tm).

In a preferred embodiment, detection methods are performed by annealing an excess of primer under conditions which favor exact hybridization, extending the hybridized primer, denaturing the extended primer, and repeating the annealing and extension reactions at least once. In a most preferred embodiment, the reaction cycle comprises a step of heat denaturation, and the polymerase is temperature stable (for example, Taq polymerase or Vent polymerase).

Preferred primer lengths are between 10 and 100 nucleotides, more preferably between 10 and 50 nucleotides, and most preferably about 30 nucleotides. Useful primers are those that hybridize adjacent a suspected mutation site, such that a single base extension at the 3′ end of the primer incorporates a nucleotide complementary to the mutant nucleotide if it is present on the template.

Preferred hybridization conditions comprise annealing temperatures about or above the Tm of the oligonucleotide primer in the reaction. The Tm of an oligonucleotide primer is determined by its length and GC content, and is calculated using one of a number of formulas known in the art. Under standard annealing conditions, a preferred formula for a primer approximately 25 nucleotides long, is Tm (° C.)=4×(Number of Gs+Number of Cs)+2×(Number of As+Number of Ts).

In a preferred reaction, the annealing and denaturation steps are performed by changing the reaction temperature. In one embodiment of the invention, the primer is annealed at about the Tm for the primer, the temperature is raised to the optimal temperature for extension, the temperature is then raised to a denaturing temperature. In a more preferred embodiment of the invention, the reaction is cycled between the annealing temperature and the denaturing temperature, and the single base extension occurs during transition from annealing to denaturing conditions.

In a preferred detection means, two or more cycles of extension are performed. In a more preferred means, between 5 and 100 cycles are performed. In a further embodiment, between 10 and 50 cycles, and most preferably about 30 cycles are performed.

In a preferred embodiment of the invention, the nucleotide added to the 3′ end of the primer in a template dependent reaction is a chain terminating nucleotide, for example a dideoxynucleotide. In a more preferred embodiment, the nucleotide is detectably labeled.

Detection methods for use in the invention may comprise conducting at least two cycles of single-base extension with a segmented primer. In a preferred embodiment, the segmented primer comprises a short first probe and a longer second probe capable of hybridizing to substantially contiguous portions of the target nucleic acid. The two probes are exposed to a sample under conditions that do not favor the hybridization of short first probe in the absence of longer second probe. Factors affecting hybridization are well known in the art and include temperature, ion concentration, pH, probe length, and probe GC content. A first probe, because of its small size, hybridizes numerous places in an average genome. For example, any given 8-mer occurs about 65,000 times in the human genome. However, an 8-mer has a low melting temperature (Tₘ) and a single base mismatch greatly exaggerates this instability. A second probe, on the other hand, is larger than the first probe and will have a higher Tₘ. A 20-mer second probe, for example, typically hybridizes with more stability than an 8-mer. However, because of the small thermodynamic differences in hybrid stability generated by single nucleotide changes, a longer probe will form a stable hybrid but will have a lower selectivity because it will tolerate nucleotide mismatches. Accordingly, under unfavorable hybridization conditions for the first probe (e.g., 10-40° C. above first probe Tₘ), the first probe hybridizes with high selectivity (i.e., hybridizes poorly to sequence with even a single mismatch), but forms unstable hybrids when it hybridizes alone (i.e., not in the presence of a second probe). The second probe will form a stable hybrid but will have a lower selectivity because of its tolerance of mismatches.

The extension reaction will not occur absent contiguous hybridization of the first and second probes. A first (proximal) probe alone is not a primer for template-based nucleic acid extension because it will not form a stable hybrid under the reaction conditions used in the assay. Preferably, the first probe comprises between about 5 and about 10 nucleotides. The first probe hybridizes adjacent to a nucleic acid suspected to be mutated. A second (distal) probe in mutation identification methods of the invention hybridizes upstream of the first probe and to a substantially contiguous region of the target (template). The second probe alone is not a primer of template-based nucleic acid extension because it comprises a 3′ non-extendible nucleotide. The second probe is larger than the first probe, and is preferably between about 15 and about 100 nucleotides in length.

Template-dependent extension takes place only when a first probe hybridizes next to a second probe. When this happens, the short first probe hybridizes immediately adjacent to the site of the suspected single base mutation. The second probe hybridizes in close proximity to the 5′ end of the first probe. The presence of the two probes together increases stability due to cooperative binding effects. Together, the two probes are recognized by polymerase as a primer. This system takes advantage of the high selectivity of a short probe and the hybridization stability imparted by a longer probe in order to generate a primer that hybridizes with the selectivity of a short probe and the stability of a long probe. Accordingly, there is essentially no false priming with segmented primers. Since the tolerance of mismatches by the longer second probe will not generate false signals, several segmented primers can be assayed in the same reaction, as long as the hybridization conditions do not permit the extension of short first probes in the absence of the corresponding longer second probes. Moreover, due to their increased selectivity for target, methods of the invention may be used to detect and identify a target nucleic acid that is available in small proportion in a sample and that would normally have to be amplified by, for example, PCR in order to be detected.

By requiring hybridization of the two probes, false positive signals are reduced or eliminated. As such, the use of segmented oligonucleotides eliminates the need for careful optimization of hybridization conditions for individual probes, as presently required in the art, and permits extensive multiplexing. Several segmented oligonucleotides can be used to probe several target sequences assayed in the same reaction, as long as the hybridization conditions do not permit stable hybridization of short first probes in the absence of the corresponding longer second probes.

The first and second probes hybridize to substantially contiguous portions of the target. For purposes of the present invention, substantially contiguous portions are those that are close enough together to allow hybridized first and second probes to function as a single probe (e.g., as a primer of nucleic acid extension). Substantially contiguous portions are preferably between zero (i.e., exactly contiguous so there is no space between the portions) nucleotides and about one nucleotide apart. A linker is preferably used where the first and second probes are separated by two or more nucleotides, provided the linker does not interfere with the assay (e.g., nucleic acid extension reaction). Such linkers are known in the art and include, for example, peptide nucleic acids, DNA binding proteins, and ligation. It has now been realized that the adjacent probes bind cooperatively so that the longer, second probe imparts stability on the shorter, first probe. However, the stability imparted by the second probe does not overcome the selectivity (i.e., intolerance of mismatches) of the first probe. Therefore, methods of the invention take advantage of the high selectivity of the short first probe and the hybridization stability imparted by the longer second probe.

First and second probes preferably are hybridized to substantially contiguous regions of target, wherein the first probe is immediately adjacent and upstream of a site of suspected mutation, for example, a single base mutation. The sample is then exposed to dideoxy nucleic acids that are complements of possible mutations at the suspected site. For example, if the wild-type nucleic acid at a known site is adenine, then dideoxy adenine, dideoxy cytosine, and dideoxy guanine are placed into the sample. Preferably, the dideoxy nucleic acids are labeled. Deoxynucleotides may alternatively be used if the reaction is stopped after the addition of a single nucleotide. Polymerase, either endogenously or exogenously supplied, catalyzes incorporation of a dideoxy base on the first probe. Detection of label indicates that a non-wild-type (i.e., mutant) base has been incorporated, and there is a mutation at the site adjacent the first probe. Alternatively, methods of the invention may be practiced when the wild-type sequence is unknown. In that case, the four common dideoxy nucleotides are differentially labeled. Appearance of more than one label in the assay described above indicates a mutation may exist.

Alternatively, a segmented oligonucleotide comprises a series of first probes, wherein sufficient stability is only obtained when all members of the segmented oligonucleotide simultaneously hybridize to substantially contiguous portions of a nucleic acid. Although short probes exhibit transient, unstable hybridization, adjacent short probes bind cooperatively and with greater stability than each individual probe. Together, a series of adjacently-hybridized first probes will have greater stability than individual probes or a subset of probes in the series. For example, in an extension reaction with a segmented primer comprising a series of three first probes (i.e., three short probes with no terminal nucleotide capable of hybridizing to a substantially contiguous portion of a nucleic acid upstream of the target nucleic acid), the concurrent hybridization of the three probes will generate sufficient cooperative stability for the three probes to prime nucleic acid extension and the short probe immediately adjacent to a suspected mutation will be extended. Thus, segmented probes comprising a series of short first probes offer the high selectivity (i.e., intolerance of mismatches) of short probes and the stability of longer probes.

Several cycles of extension reactions preferably are conducted in order to amplify the assay signal. Extension reactions are conducted in the presence of an excess of first and second probes, labeled dNTPs or ddNTPs, and heat-stable polymerase. Once an extension reaction is completed, the first and second probes bound to target nucleic acids are dissociated by heating the reaction mixture above the melting temperature of the hybrids. The reaction mixture is then cooled below the melting temperature of the hybrids and first and second probes permitted to associate with target nucleic acids for another extension reaction. In a preferred embodiment, 10 to 50 cycles of extension reactions are conducted. In a most preferred embodiment, 30 cycles of extension reactions are conducted.

Labeled ddNTPs or dNTPs preferably comprise a “detection moiety” which facilitates detection of the extended primers, or extended short first probes in a segmented primer reaction. Detection moieties are selected from the group consisting of fluorescent, luminescent or radioactive labels, enzymes, haptens, and other chemical tags such as biotin which allow for easy detection of labeled extension products. Fluorescent labels such as the dansyl group, fluorescein and substituted fluorescein derivatives, acridine derivatives, coumarin derivatives, pthalocyanines, tetramethylrhodamine, Texas Red®, 9-(carboxyethyl)-3-hydroxy-6-oxo-6H-xanthenes, DABCYL® and BODIPY® (Molecular Probes, Eugene, Oreg.), for example, are particularly advantageous for the methods described herein. Such labels are routinely used with automated instrumentation for simultaneous high throughput analysis of multiple samples.

In a preferred embodiment, primers or first probes comprise a “separation moiety.” Such separation moiety is, for example, hapten, biotin, or digoxigenin. These primers or first probes, comprising a separation moiety, are isolated from the reaction mixture by immobilization on a solid-phase matrix having affinity for the separation moiety (e.g., coated with anti-hapten, avidin, streptavidin, or anti-digoxigenin). Non-limiting examples of matrices suitable for use in the present invention include nitrocellulose or nylon filters, glass beads, magnetic beads coated with agents for affinity capture, treated or untreated microtiter plates, and the like.

In a preferred embodiment, the separation moiety is incorporated in the labeled ddNTPs or dNTPs. By denaturing hybridized primers or probes, and immobilizing primers or first probes extended with a labeled ddNTP or dNTP to a solid matrix, labeled primers or labeled first probes are isolated from unextended primers or unextended first probes and second probes, and primers or first probes extended with an unlabeled ddNTPs by one or more washing steps.

In an alternative preferred embodiment, the separation moiety is incorporated in the primers or first probes, provided the separation moiety does not interfere with the first primer's or probe's ability to hybridize with template and be extended. Eluted primers or first probes are immobilized to a solid support and can be isolated from eluted second probes by one or more washing steps.

Alternatively, the presence of primers or first probes that have been extended with a labeled terminal nucleotide may be determined without eluting hybridized primers or probes. The methods for detection will depend upon the label or tag incorporated into the primers or first probes. For example, radioactively labeled or chemiluminescent first probes that have bound to the target nucleic acid can be detected by exposure of the filter to X-ray film. Alternatively, primers or first probes containing a fluorescent label can be detected by excitation with a laser or lamp-based system at the specific absorption wavelength of the fluorescent reporter.

In an alternative embodiment, the bound primers or first and second probes are eluted from a matrix-bound target nucleic acid (see below). Elution may be accomplished by any means known in the art that destabilizes nucleic acid hybrids (i.e., lowering salt, raising temperature, exposure to formamide, alkali, etc.). In a preferred embodiment, the bound oligonucleotide probes are eluted by incubating the target nucleic acid-segmented primer complexes in water, and heating the reaction above the melting temperature of the hybrids.

Deoxynucleotides may be used as the detectable single extended base in any of the reactions described above that require single base extension. However, in such methods, the extension reaction must be stopped after addition of the single deoxynucleotide. Such methods may be employed regardless of whether a specific mutation is known (i.e., C→G). Moreover, the extension reaction need not be terminated after the addition of only one deoxynucleotide if only one labeled species of deoxynucleotide is made available in the sample for detection of the single base mutation. This method may actually enhance signal if there is a nucleotide repeat including the interrogated single base position.

In a preferred embodiment, target nucleic acids are immobilized to a solid support prior to exposing the target nucleic acids to primers or segmented primers and conducting an extension reaction. Once the nucleic acid samples are immobilized, the samples are washed to remove non-immobilized materials. The nucleic acid samples are then exposed to one or more set of primers or segmented primers according to the invention. Once the single-base extension reaction is completed, the primers or first probes extended with a labeled ddNTP or dNTP are preferably isolated from unextended probes and probes extended with an unlabeled ddNTPs or dNTP. Bound primers or first and second probes are eluted from the support-bound target nucleic acid. Elution may be accomplished by any means known in the art that destabilizes nucleic acid hybrids (i.e., lowering salt, raising temperature, exposure to formamide, alkali, etc.). In a preferred embodiment, the first and second probes bound to target nucleic acids are dissociated by incubating the target nucleic acid-segmented primer complexes in water, and heating the reaction above the melting temperature of the hybrids and the extended first probes are isolated. In an alternative preferred embodiment, the extension reaction is conducted in an aqueous solution. Once the single-base extension reaction is completed, the oligonucleotide probes are dissociated from target nucleic acids and the extended first probes are isolated. In an alternative embodiment, the nucleic acids remain in aqueous phase.

Finally, methods of the invention comprise isolating and sequencing the extended first probes. A “separation moiety” such as, for example, hapten, biotin, or digoxigenin is used for the isolation of extended first probes. In a preferred embodiment, first probes comprising a separation moiety are immobilized to a solid support having affinity for the separation moiety (e.g., coated with anti-hapten, avidin, streptavidin, or anti-digoxigenin). Non-limiting examples of supports suitable for use in the present invention include nitrocellulose or nylon filters, glass beads, magnetic beads coated with agents for affinity capture, treated or untreated microtiter plates, and the like.

What is claimed is:

1. A method for diagnosing the disease state of a patient, the method comprising the steps of:
(a) combining tissue or body fluid samples obtained from a plurality of patients, thereby to form a combined sample;
(b) analyzing said combined sample for the presence of a disease marker; and
(c) diagnosing the disease status of each member of said plurality by conducting at least one step selected from the group consisting of:
(1) Identifying each member of said plurality as being healthy if no disease marker is detected in said analyzing step; and
(2) Serially analyzing patient samples if a disease marker is detected in said analyzing step;
thereby to diagnose the disease state of each member of said plurality.

2. The method of claim 1, wherein said disease marker is a nucleic acid.

3. The method of claim 2, wherein said nucleic acid is a mutation.

4. The method of claim 3, wherein said analyzing step comprises
(a) exposing said combined sample to a first nucleic acid probe capable of specific hybridization with a nucleic acid known or suspected to be mutated in diseased cells,
(b) exposing said combined sample to a nucleic acid probe capable of specific hybridization with a nucleic acid known not to be mutated in disease cells;
(c) enumerating a number of first and second probes that hybridize in said combined sample; and
(d) determining whether a statistically-significant difference exists between the number of first and second probes.

5. The method of claim 2, wherein said analyzing step comprises the steps of
(a) annealing an oligonucleotide primer to a nucleic acid sample under conditions that promote exact complementary hybridization between said primer and a portion of a nucleic acid in said combined sample;
(b) extending said primer by a single base; and
(c) identifying said single base.

6. The method of claim 5, further comprising the step of determining whether said single base is a known polymorphic variant indicative of disease.

7. The method of claim 2, wherein said analyzing step comprises
(a) exposing said combined sample to a nucleic acid primer under conditions that promote hybridization of said probe to a nucleic acid region immediately downstream of a single nucleotide polymorphic locus;
(b) exposing said sample to at least four different chain-terminating nucleic acids under conditions that promote extension of said primer;
(c) isolating extended primer in either from primer that has not been extended;
(d) determining a number of each unique chain terminating nucleic acid attached to an extended primer; and
(e) determining if a statistically-significant difference occurs between said numbers.

8. The method of claim 1, wherein said tissue or body fluid sample is selected from the group consisting of sputum, stool, blood, cerebrospinal fluid; biopsy tissue, urine, semen, lymph, and pap smear.

9. The method of claim 1, wherein said disease is selected from the group consisting of cancer, diabetes, amyotropic lateral sclerosis, AIDS, Alzheimer's disease, and parasitic diseases.

10. The method of claim 1, wherein said plurality comprises from 2 to about 25 patients.

11. The method of claim 1, wherein said plurality comprises 100 patients.

12. The method of claim 1, wherein said plurality comprises 1000 patients.

13. The method of claim 3, wherein said mutation is selected from the group consisting of a point mutation, loss of heterozygosity, a rearrangement, a deletion, and inversion, and a translocation.

14. The method of claim 1, wherein said DNA is isolated from said combined sample prior to said analyzing step.
